(19) United States
(12) Patent Application Publication
(10) Pub. No.: US 2009/0149761 A1
(43) Pub. Date: Jun. 11, 2009

(54) PHOTOACOUSTIC IMAGING METHOD

(75) Inventors: Hans Zou, Windsor, NJ (US); Ladislav Jankovic, Wappingers Falls, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/302,346

(22) PCT Filed: Apr. 11, 2007
(86) PCT No.: PCT/IB2007/051298

§ 371 Date: Nov. 25, 2008

Related U.S. Application Data
(60) Provisional application No. 60/803,171, filed on May 25, 2006.

(51) Int. Cl. A61B 6/00

(52) U.S. Cl. 600/476

(57) ABSTRACT

This invention discloses a method to position, identify and characterize a photoacoustic source in a complex environment. This method isolates individual acoustic responses from interferences by spectral analysis and filtering and locates primary acoustic sources by applying beam-forming to decomposed acoustic responses. The photon-absorbing structure of a tissue can be constructed with primary source parameters.

The invention relates to a photoacoustic imaging method for specimens having one or more photoacoustic origins.

In the last couple of decades, various non-invasive diagnostic techniques such as X-ray imaging, magnetic resonance imaging (MRI), ultrasound, positron emission tomography (PET), optical coherence tomography (OCT), elastic and diffuse reflectance, photoacoustics, fluorescence, Raman scattering, etc., have been employed to diagnose malignant tumors in vivo. Depending on the method employed to differentiate between normal and tumorous tissues, these different techniques can be classified as either morphological-based or chemical-based analyses.

Morphological-based methods such as X-ray, OCT, and ultrasound differentiate normal and tumorous tissues based on differences in densities between cancerous and non-cancerous tissues or on their water content. Because these techniques differentiate tissues based on tissue density, they are under certain conditions unable to accurately distinguish between dense healthy tissues and tumorous tissues.

Chemical-based techniques (i.e., fluorescence spectroscopy, etc.), on the other hand, differentiate normal and tumorous tissues by measuring differences in chemical composition (e.g., hemoglobin content and oxygenation level etc.). In order to perform such analyses, ultraviolet or blue light (300 nm to 450 nm) is typically required for excitation of the tissue, as these wavelengths have sufficient energy to excite the various chemical species being interrogated. However, the applicability of fluorescence spectroscopy for tumor diagnosis is dramatically limited in view of shortcomings associated with its use; these include low signal associated with light penetration depth, poor resolution, use of PMTs, background signal, filtering light out and the need for a dark chamber conditions.

Photoacoustic tomography of a biological tissue is based on the photoacoustic effect that takes place when photons are absorbed by a tissue structure. Upon absorption, photon energy is converted to heat, which in turn causes local thermal expansion. This expansion generates a thermoelastic pressure transient (shock wave) that represents the absorbing structures of the tissue. Photoacoustic waves can be detected by one or more receivers (transducers) and be used to construct the image of the absorbing structure. Because of their differences in optical absorption thermal elasticity and even size of the absorbing volume, different biological tissues have different photoacoustic responses. Photoacoustic imaging is, for example, disclosed in U.S. Patent Application Numbers 20050070803 published on Mar. 31, 2005 and 20050004458 published on Jan. 6, 2005.

However, problems still persist with these techniques. Specifically with regard to using photoacoustics for imaging a real biological target, a photon-absorbing structure is often complicated, making reconstruction of a photoacoustic image difficult. First, multiple photon-absorbing sources made of biological tissues of different properties may coexist. Second, photoacoustic waves may experience multiple bounces following various paths before they reach the transducer. Third, interference between these multiple sources and echoes may distort original signals in a very complicated way. For general clinical diagnosis, photoacoustic imaging is preferred to operate in a reflection mode, where both light source and transducer are on the same side of a target. In this case, the interference problem become worse because of stronger disturbance along the light-incident path.

According to this invention, construction of a photoacoustic image is accomplished by applying beamforming to time resolved photoacoustic signals that are sorted according to their spectral distributions. In one embodiment, signals from each transducer are analyzed for spectral distribution and decomposed into individual photoacoustic responses based on their spectral distribution. Then, these responses are sorted in groups according to their similarities. A photon absorbing (or photoacoustic) origin is located and characterized by applying the beamforming algorithm to the responses in the same group. The entire photon-absorbing structure is reconstructed by assembling individual photoacoustic origins. To facilitate component analysis and sorting, a scalable (in terms of absorbing coefficient, geometrical size and thermo-elasticity) mode of photoacoustic response of biological tissues can be applied.

It is an object of this invention to provide a method for performing spectral imaging for a specimen having one or more photoacoustic origins comprising: generating photon excitation in the specimen; detecting photoacoustic responses resulting from the excitation; sorting the responses into groups having similar spectral distribution; applying a beam-forming algorithm to the responses in the same group to locate and characterize each photoacoustic origin; and forming a spectral image by assembling the individual photoacoustic origins.

Another object is to provide a method wherein the generation step comprises irradiating the specimen with pulsed laser light within a predetermined range of wavelengths.

Another object is to provide a method wherein the detection step comprises detecting the photoacoustic responses resulting from the excitation using one or more transducers.

Another object is to provide a method further comprising analyzing signals received from each transducer for spectral distribution and decomposing the signals into individual photoacoustic responses based on their spectral distribution.

Another object is to provide a method wherein the specimen is a biological tissue.

Another object is to provide a method wherein the photoacoustic origin is a tumor, blood vessel or cyst.

These and other aspects of the invention are explained in more detail with reference to the following embodiments and with reference to the figures.

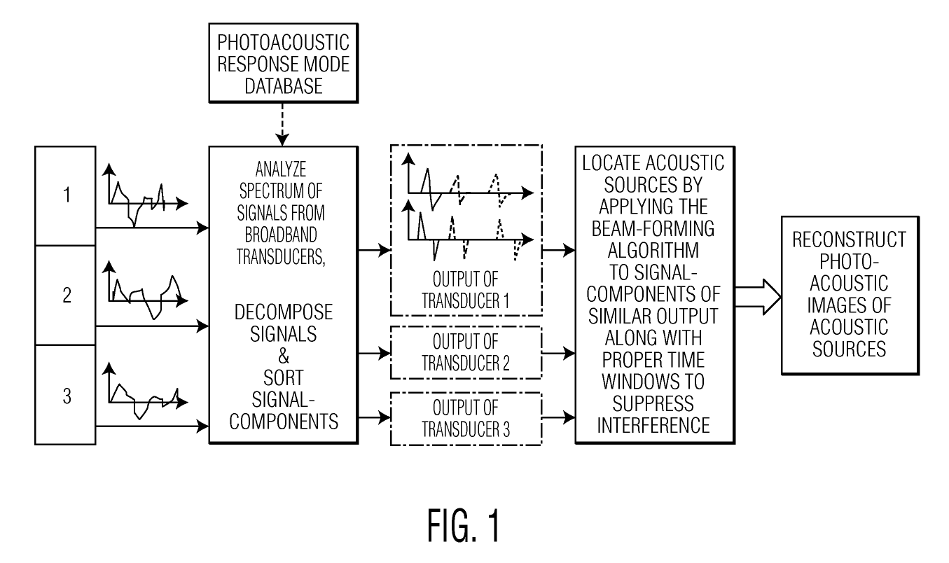

FIG. 1 is a block diagram of reconstruction of the photon-absorbing structure of a biological tissue For illustration purpose, only three transducers are drawn, the time-resolved decomposed signal components are only symbolically indicated in the output box of transducer 1. A photo-acoustic response mode database can be used to decompose signals.

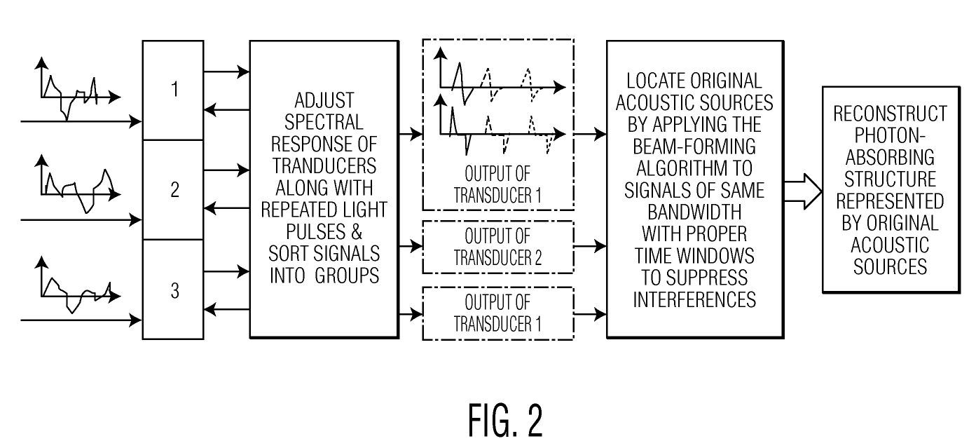

FIG. 2 is a block diagram of reconstruction of both photon-absorbing structure and environmental structure of a biological tissue. For illustration purpose, only three transducers are drawn, the time-resolved decomposed signal components are only symbolically indicated in the output box of transducer 1.

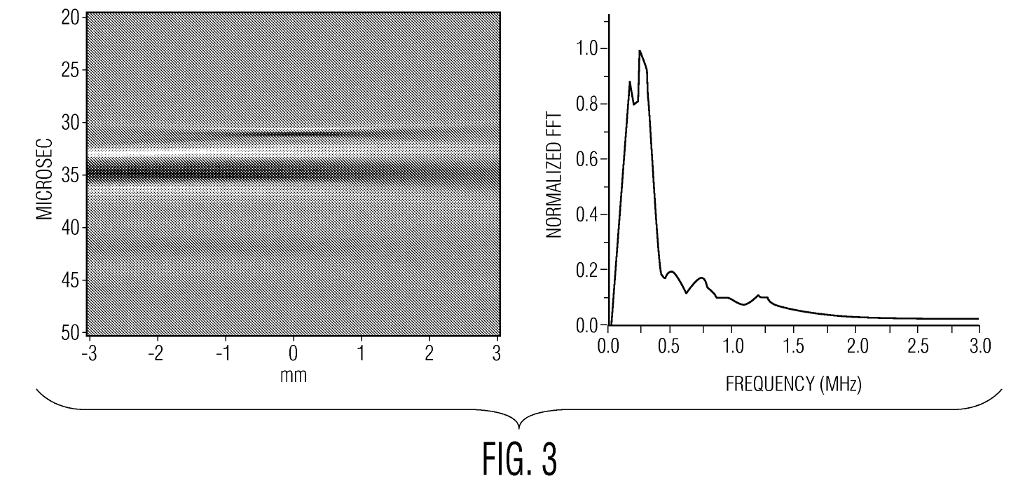

FIG. 3 is a (left) compound image of two closely spaced tubes (0.5 and 3 mm diameter). (right) Time domain Fourier transform of the image (shown up to 3.0 MHz shown).

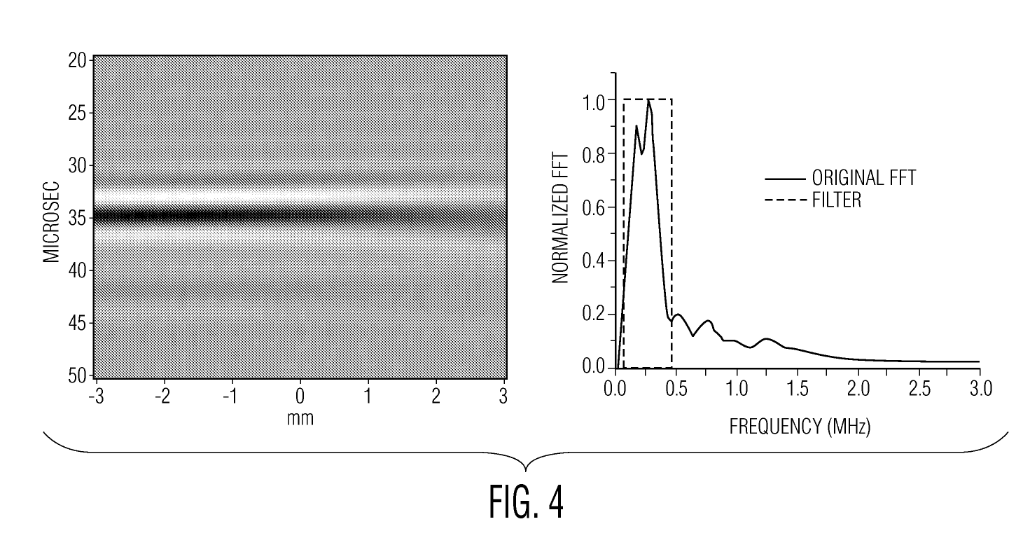

FIG. 4 is a (right) spectral profile of the initial, unfiltered image, and the filter used. (left) Image after applying the bandpass filter.

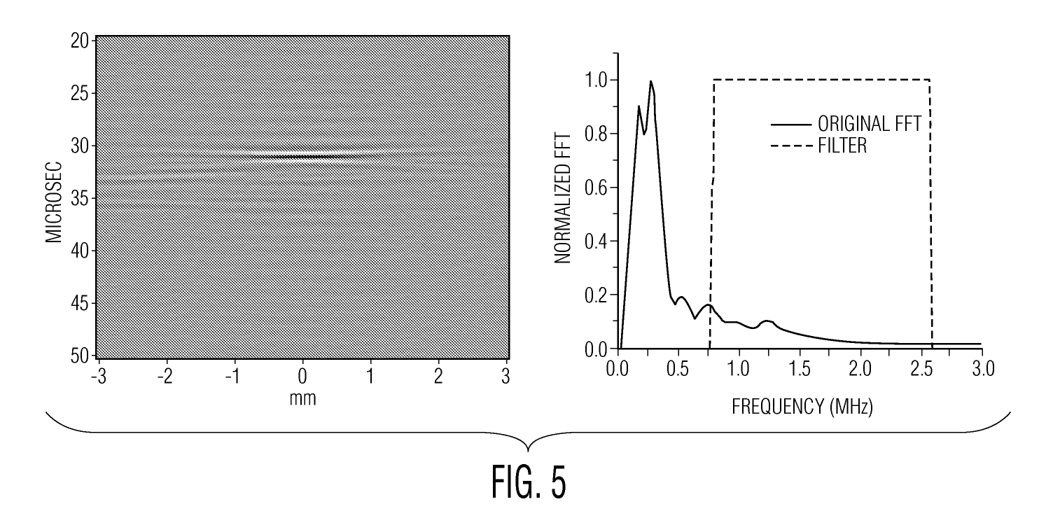

FIG. 5 is a (right) spectral profile of the initial, unfiltered image, and the filter used. (left) Image after applying the bandpass filter.

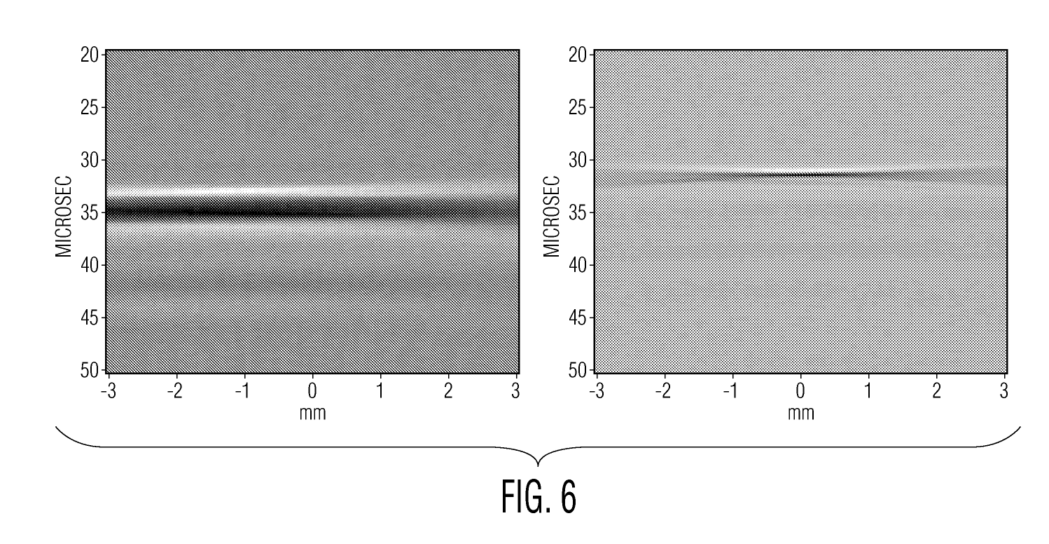

FIG. 6 shows original aligned rf-data maps.

In recent years a broad interest is present in developing new techniques for non-invasive imaging of blood vessels and blood containing structures, such as tumors, in tissue. The purpose is to detect early or precancer that are undetectable with existing techniques since increased blood supply and capillary growth takes place in the early stage of all epithelial cancers.

Photoacoustics is a technique that is based on the generation of sound waves by modulated or pulsed optical radiation. The efficiency of sound generation is higher for pulsed than for modulated radiation. In pulsed photoacoustics a short laser pulse heats absorbers inside the tissue, producing a temperature rise proportional to the deposited energy. The light pulse is so short that adiabatic heating of the absorber occurs, resulting in a sudden pressure rise. The resulting pressure wave (acoustic wave) will propagate through the tissue and can be detected at the tissue surface. From the time this pressure wave needs to reach the tissue surface (detector position), the position of the photoacoustic source can be determined. Detection of photoacoustic waves can be carried out using piezoelectric or optical interference methods.

The difference in absorption between tissue-constituents (i.e., photoacoustic origins) and the tissue (i.e., specimen) itself can be used to reveal information about these constituents. A well-known absorber in tissue is blood (hemoglobin), which enables localization and monitoring of blood concentrations (vessels, tumors) in tissues. Instead of using blood as an absorber, also other tissue chromophores such as glucose can be used.

Various purely optical diagnostic techniques are based on light scattering in tissue. In highly scattering media, like dermal tissue, the scattering coefficient not only determines the penetration depth, but also limits the resolution that can be achieved by the technique. With photoacoustic signal generation, the amplitude depends on the local fluence only. The preceding light path of the photon, caused by scattering, is not relevant. For this reason, the spatial resolution is not influenced by tissue scattering and it has been shown that photoacoustics is a promising technique to visualize absorbing structures in tissue-like media. (See. Proceedings of the SPIE—The International Society for Optical Engineering-2004-SPIE-Int. Soc. Opt. Eng-USA, CONF-Photon Plus Ultrasound: Imaging and Sensing, 25-26 Jan. 2004,-San Jose, Calif., USA, AU-Kolkman R G M; Huisjes A; Sipahto R I; Steenbergen W; van Leeuwen T G, AUAF-Fac. of Sci. & Technol., Twenty Univ., Enschede; Netherlands, IRN-ISSN 0277-786X, VOL-5320, NR-1 PG-16-20.)

The proposed invention is directed to a method to position, identify and characterize a photo-acoustic source in a complex environment. This method isolates individual acoustic responses (i.e., acoustic origins) from interferences by spectral analysis and filtering and locates primary acoustic sources by applying beam-forming to decomposed acoustic responses. The photon-absorbing structure of a tissue can be constructed with primary source parameters.

Physically, beam-forming is to locate a signal source by analyzing time-dependent signals received by an array of detectors. Assuming transmission speed of the signal is the same in all directions, this speed times the elapsed time of the signal received by each detector determines the distance from the source to the corresponding detector. In principle, three detectors at different positions are sufficient to locate the source position.

Mathematically, the task of beam-forming is to find out the coordinates of the merging point of three vectors with known start point coordinates (in this case, the detector position) and length (in this case, the distance) of each vector. It is straightforward to locate a point source position in a homogenous medium by applying beam-forming technique.

In order to reconstruct a photoacoustic image from the measured rf waveforms one can use the modified beam-forming algorithms, such as delay-and-sum beam-forming and Fourier beam-forming, which are widely known in diagnostic ultrasound (particularly the delay-and-sum). The modification is needed since in photoacoustics the beam-forming is performed based on the signals originating from practically the entire tissue volume, rather than from a number of the narrow slices, like in the diagnostic ultrasound.

A general form of the delay-and-sum photoacoustic beamformer (without spectral filtering) can be expressed as:

$$s ( t , x ) = {\sum }_{{i}_{( elements )}} {w}_{i} ( t , x ) {p}_{i} ( t - {t}_{i} ( x ) )$$

Here (t,x) is a point in the tissue cross-section of interest, pᵢ(t) is per-channel RF signal, tᵢ(x) is time delay applied on each channel, wᵢ(t, x) performs both receive aperture apodization and time gain compensation, and s(t, x) represents one sample point in the reconstructed image.

A Fourier beam-forming algorithm has been discussed in the references (K. P. Kostli, D. Frauchiger, J. J. Niederhauser, G. Paltauf, H. P. Weber, and M. Frenz, “Optoacoustic imaging using a three-dimensional reconstruction algorithm,” IEEE J. Sel. Topics Quantum Electron., vol. 7, no. 6, pp. 918-923, November-December 2001.) and (K. P. Kostli and P. C. Beard, “Two-dimensional photoacoustic imaging by use of fourier-transform image reconstruction and a detector with an anisotropic response,” Appl. Opt., vol. 42, no. 10, pp. 1899-1908, 2003.).

In the proposed method one would apply an appropriate filtering algorithm on the waveforms pᵢ(t), sort and group the altered [pᵢ(t)]ₘ waveforms (here m is the group number). The above discussed beam-forming algorithm is consequently applied on the [pᵢ(t)]ₘ instead of pᵢ(t). The filtering might be such as bandpass filtering, wavelet filtering or based on some other separation role.

According to this invention, construction of a photo-acoustic image is by applying beam-forming to time resolved photo-acoustic signals that are sorted according to their spectral distributions. In one illustrative aspect, signals from each transducer are analyzed for spectral distribution and decomposed into individual photo-acoustic responses based on their spectral distribution. Then, these responses are sorted in groups according to their similarities. A photon absorbing origin is located and characterized by applying the beam-forming algorithm to the responses in the same group. The entire photon-absorbing structure is reconstructed by assembling individual photo-acoustic origins. To facilitate component analysis and sorting, a scalable (in terms of absorbing coefficient, geometrical size and thermo-elasticity) mode of photo-acoustic response of biological tissues can be applied. Examples 1 and 2 below illustrate through block diagrams how a photoacoustic image is reconstructed or formed in accordance with the invention.

### EXAMPLE 1

Reconstruction of a photo-acoustic image by applying the beam-forming algorithm to decomposed photo-acoustic responses. FIG. 1 shows the block diagram of the first example of the invention.

### EXAMPLE 2

Reconstruction of a photon-absorbing image represented by original acoustic sources by applying the beam-forming algorithm to filtered photoacoustic responses. FIG. 2 shows the block diagram of the second example of the invention.

In photoacoustic imaging of biological tissues, the characteristics of detected acoustic signals is typically related to the physical properties of imaged objects.

A typical example of such biological objects would be a blood vessel or a cyst. They can be substantially different in size, and positioned in a way that is difficult to detect them separately. Due to the fact that spectral property of photoacoustic signal varies with the size of a photoacoustic source one can use spectral filtering in order to separate multiple photoacoustic sources, which can normally not be separated. An example of spectral filtering is provided below in Example 3.

### EXAMPLE 3

Two ink filled tubes, ˜0.5 mm and ˜3 mm diameter, were used in the experiment. Each tube immersed in water was illuminated with 532 nm light from a 10 Hz repeat-rate, pulsed Nd:YAG laser (pulse duration 5 ns). The photoacoustic signal from each tube was recorded separately with a 2.25 MHz transducer. These separately recorded photoacoustic images of two tubes were merged later to mimic the image of two closely spaced objects of different sizes.

FIG. 3 shows the compound image of two tubes and its spectral content. The image represents acoustic rf-lines, which were put together into an aligned rf-data map with the receiving transducer position as the horizontal axis, and time of flight as the vertical one. Such rf-data sequence map would be later used in a beam-forming algorithm to generate an image of the photoacoustic objects. Here we limit the discussion to rf-data maps only, which are in fact pre-beamformed. In the frequency distribution map there is very little contribution from the high frequencies. It is because the measured signal bandwidth was limited by that of the transducer and the acquisition process, which together act as a bandpass/lowpass filter. Even so, the available frequency distribution is sufficient to demonstrate our objective of using spectral filtering to resolve spatially overlapped objects of different sizes.

Bandpass filters, as shown in the FIG. 4 (right) and FIG. 5 (right) were applied to the merged rf-data map (FIG. 3) separately. The results are shown in FIG. 4 (left) and FIG. 5 (left), respectively. Each filtering intensifies one of the objects and suppresses the other one, because two objects have different spectral content. Resolving objects based on their spectral content is photoacoustic related and cannot be used in standard pulse-echo ultrasound imaging. It should be noted that the bandpass filter used in the example is just for demonstration purpose. A filter with a profile other than gate function can be used to optimize filtering specificity. For example, if the spectral distribution of a specific feature is known, a filter matching the distribution profile of this feature can be applied to the raw data.

SNR (i.e. signal to noise ratio) in the given examples (FIG. 4 and FIG. 5) is lower as compared with original data maps in FIG. 6. In order to increase the SNR, transducers and data acquisition with wide bandwidth and more accurate filtering would be required.

This invention will simplify the process of identifying different photoacoustic sources (i.e., Photoacoustic origins) and significantly improve the quality of image reconstruction of photon-absorbing structures of a biological tissue (i.e., specimen). Implementation of this invention will allow a clinical photoacoustic imaging device to be used for in vivo diagnosis of complicated biological tissues, such as a tumor detection and therapy monitoring.

While the present invention has been described with respect to specific embodiments thereof, it will be recognized by those of ordinary skill in the art that many modifications, enhancements, and/or changes can be achieved without departing from the spirit and scope of the invention. Therefore, it is manifestly intended that the invention be limited only by the scope of the claims and equivalents thereof.

1. A method for performing spectral imaging for a specimen having one or more photoacoustic origins comprising:
generating photon excitation in the specimen;
detecting photoacoustic responses resulting from the excitation;
sorting the responses into groups having similar spectral distribution;
applying a beam-forming algorithm to the responses in the same group to locate and characterize each photoacoustic origin;
and forming a spectral image by assembling the individual photoacoustic origins.

2. The method of claim 1 wherein the generation step comprises irradiating the specimen with pulsed laser light within a predetermined wavelength range from about 500 nm to 1200 nm.

3. The method of claim 1 wherein the detection step comprises detecting the photoacoustic responses resulting from the excitation using one or more transducers.

4. The method of claim 3 further comprising analyzing signals received from each transducer for spectral distribution and decomposing the signals into individual photoacoustic responses based on their spectral distribution.

5. The method of claim 1 wherein the specimen is a biological tissue.

6. The method of claim 1 wherein the photoacoustic origin is a tumor, blood vessel or cyst.
